(19) United States
(12) Patent Application Publication
(10) Pub. No.: US 2025/0177502 A1
(43) Pub. Date: Jun. 5, 2025

(54) THROMBIN AND FIBRINOGEN AS TARGETS FOR ATOPIC DERMATITIS DIAGNOSIS AND TREATMENT

(71) Applicant: Children's Hospital Medical Center, Cincinnati, OH (US)

(72) Inventors: Michael SHERENIAN, Cincinnati, OH (US); Gurjit HERSHEY, Cincinnati, OH (US); Joseph PALUMBO, Cincinnati, OH (US)

(21) Appl. No.: 18/727,424

(22) PCT Filed: Jan. 13, 2023
(86) PCT No.: PCT/US2023/060690

§ 371 Date: Jul. 9, 2024

Related U.S. Application Data
(60) Provisional application No. 63/299,269, filed on Jan. 13, 2022.

(51) Int. Cl. A61K 38/58; A61K 9/00; A61K 31/4439; A61K 47/26; A61P 17/00; G01N 33/86

(52) U.S. Cl. CPC A61K 38/58; A61K 9/0014; A61K 31/4439; A61K 47/26; A61P 17/00; G01N 33/86; G01N 2333/75; G01N 2333/974; G01N 2800/202

(57) ABSTRACT

Methods for diagnosing atopic dermatitis (AD) by using thrombin and/or fibrinogen as biomarkers to determine the presence or severity of AD. Also provided are methods for treating AD by administering to a subject an effective amount of a thrombin inhibitor.

### CROSS REFERENCE TO RELATED APPLICATIONS

This application claims the benefit of the filing date of U.S. Provisional Application No. 63/299,269, filed on Jan. 13, 2022, the content of which is herein incorporated by reference in its entirety.

### GOVERNMENT RIGHTS

This invention was made with government support under U19AI070235 and 5K12HD28827-27, awarded by the National Institutes of Health. The United States government has certain rights in the invention.

### BACKGROUND OF THE INVENTION

Atopic dermatitis (AD), also known as eczema, is a long-term type of inflammation of the skin that results in itchy, red, swollen, and cracked skin. AD most often affects infants and young children, but it can continue into adulthood or first show up later in life, affecting approximately 17.8 million persons in the United States. The exact cause of the condition is still unclear. In spite of its high clinical significance, the current strategy for diagnosis of AD depends largely on obtaining the patient's family history and visual assessment of the skin, and effective treatment is limited.

It is therefore of great interest to have a better understanding of the pathogenesis of this disease and to develop new, effective diagnosis and treatment methods for AD.

### SUMMARY OF THE INVENTION

The present disclosure is based, at least in part, on the discovery of mechanistic association between plasma thrombin generation and atopic dermatitis (AD) pathogenesis and that thrombin and fibrinogen can be used as effective diagnostic biomarkers and/or treatment targets for AD.

Accordingly, in one aspect, the present disclosure provides a method for treating atopic dermatitis in a subject (e.g., a human subject), the method comprising: administering to a subject in need thereof an effective amount of a composition, which comprises a therapeutic agent inhibiting plasma thrombin and a pharmaceutically acceptable carrier.

In some embodiments, the subject is a human pediatric patient having atopic dermatitis. In some embodiments, the subject has moderate-to-severe atopic dermatitis. In some examples, the subject having moderate-to-severe atopic dermatitis can be diagnosed by a SCORing Atopic Dermatitis (SCORAD) index scoring system, an Eczema Area and Severity Index (EASI) scoring system, or a combination thereof.

In some embodiments, the subject has an increased level of total plasma thrombin or peak plasma thrombin as compared to a reference value for total plasma thrombin or peak plasma thrombin. In some examples, the level of total plasma thrombin or peak plasma thrombin is measured by a thrombin generation assay (TGA).

In some embodiments, the subject has an increased level of plasma fibrinogen as compared to a reference value for plasma fibrinogen. In some examples, the level of plasma fibrinogen is measured by an immunohistochemistry (IHC) staining assay or an enzyme-linked immunosorbent assay (ELISA).

In some embodiments, the subject has an increased level of transepidermal water loss (TEWL) as compared to a reference value for TEWL. In some examples, the level of transepidermal water loss (TEWL) is measured using a Dermalab instrument.

In some embodiments, the subject has a skin barrier dysfunction. Alternatively or in addition, the subject is free of a genetic thrombophilic disorder associated with a Factor V Leiden (FFL, rs6025) mutation or a Prothrombin G20210A (rs1799963) mutation.

In some embodiments, the therapeutic agent is a direct thrombin inhibitor. Examples include, but are not limited to, dabigatran, bivalirudin, argatroban, hirudin, lepirudin, desirudin, argatroban, inogatran, melagatran, and ximelagatran. In some embodiments, the composition can be administered orally, parenterally (e.g., intravenously), or topically.

In another aspect, the present disclosure provides method for diagnosing atopic dermatitis in a subject (e.g., in a human subject), the method comprising: (a) measuring a level of a biomarker in one or more biological samples from a subject suspected of having atopic dermatitis; (b) comparing the level of the biomarker in the biological sample(s) with a reference value for the biomarker; and (c) determining presence or severity of atopic dermatitis in the subject based on the comparing results of (b). The biomarker can be thrombin, fibrinogen, transepidermal water loss (TEWL), or a combination thereof. An elevated level of the biomarker in the biological sample(s) relative to the reference value indicates that the subject has atopic dermatitis or has moderate-to-severe atopic dermatitis. In some instances, the biological sample(s) comprises blood sample(s), skin sample(s), or a combination thereof.

In some embodiments, the biomarker comprises total plasma thrombin, peak plasma thrombin, or a combination thereof. In some examples, the level of the total plasma thrombin and/or peak plasma thrombin is measured in a blood sample of the subject. Alternatively or in addition, the biomarker comprises plasma fibrinogen. In some examples, the level of the total plasma thrombin and/or peak plasma thrombin is measured by a thrombin generation assay (TGA).

In some embodiments, the biomarker comprises fibrinogen. In some examples, the level of fibrinogen is measured in a skin sample of the subject. For example, the level of the fibrinogen (e.g., in a skin sample) can be measured by an immunohistochemistry (IHC) staining assay, an enzyme-linked immunosorbent assay (ELISA), or a combination thereof.

In some embodiments, the biomarker comprises transepidermal water loss (TEWL). In some examples, the level of transepidermal water loss (TEWL) is measured using a Dermalab instrument in a skin sample of the subject.

Any of the diagnostic methods disclosed herein may further comprise assessing atopic dermatitis severity in a subject who has been determined to have atopic dermatitis by the method disclosed herein using a SCORing Atopic Dermatitis (SCORAD) index scoring system, an Eczema Area and Severity Index (EASI) scoring system, or a combination thereof.

In some embodiments, the subject is a human pediatric subject. In some embodiments, the subject has a skin barrier dysfunction. In some embodiments, the subject is free of a genetic thrombophilic disorder associated with a Factor V Leiden (FFL, rs6025) mutation or a Prothrombin G20210A (rs1799963) mutation.

Any of the diagnostic method disclosed herein may the comprise treating the subject who is determined as having atopic dermatitis in step (c) a therapeutic agent inhibiting plasma thrombin. Any treatment methods as disclosed herein may be applied to such a subject.

Moreover, provided herein is a lyophilized formulation, which is lyophilized from an aqueous solution comprising about 40-60 mg/ml bivalirudin, about 20-30 mg/ml mannitol, and a pH of about 5-6. In some examples, the aqueous solution comprises 50 mg/ml of bivalirudin and 25 mg/ml mannitol. In some examples, the aqueous solution comprises NaOH for pH adjustment. The lyophilized formulation may be reconstituted with water or a buffering agent to form an aqueous formulation comprising about 40-60 mg/ml bivalirudin (e.g., 50 mg/ml), about 20-30 mg/ml mannitol (e.g., 25 mg/ml), and having a pH of about 7. Such an aqueous formulation may be used in any of the method disclosed herein for topical administration.

In other aspects, the present disclosure features a method for treating a skin disorder in a subject, the method comprising: (i) providing any of the lyophilized formulation as disclosed herein, (ii) reconstituting the lyophilized formulation with water or a buffering agent to produce a final aqueous formulation comprising about 40-60 mg/ml bivalirudin, about 20-30 mg/ml mannitol, and having a pH of about 7; and (iii) administering the final aqueous formulation topically to a subject who needs the treatment (e.g., those disclosed herein). In some examples, the final aqueous formulation comprises 50 mg/ml bivalirudin and 25 mg/ml mannitol and has a pH of about 7. In some examples, the subject is a human patient having atopic dermatitis. In one example, the human patient is a pediatric patient.

Also within the scope of the present disclosure are thrombin inhibitors (e.g., those disclosed herein) or pharmaceutical compositions comprising such (e.g., any of the bivalirudin-containing aqueous or lyophilized formulations disclosed herein) for use in treating a skin disorder such as atopic dermatitis or for manufacturing a medicament for use in treatment of the skin disorder.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1A shows the thrombin generation assay (TGA) overview. FIGS. 1B and 1C show the correlation between citrate and EDTA samples (FIG. 1B) endogenous thrombin potential (total thrombin) and (FIG. 1C) peak thrombin generated. Note that for both Endogenous thrombin potential and peak thrombin there is a strong correlation between the two anticoagulants for each parameter.

FIGS. 2A-2E are graphs showing the thrombin generation assay results in children without and with AD. FIG. 2A shows lag phase, time to thrombin clot initiation; FIG. 2B shows peak thrombin; FIG. 2C shows peak time, time to peak thrombin; FIG. 2D shows velocity index, rate of thrombin generation; and FIG. 2E shows area under the curve (AUC), total thrombin generation. Note significant differences in children with AD compared to those without in lag phase, peak thrombin, peak time, and the velocity index.

FIGS. 3A-3E are graphs showing the thrombin generation assay results in children with no AD, with mild AD, and with moderate-to-severe AD. FIG. 3A shows AUC, total thrombin generation; FIG. 3B shows peak thrombin; FIG. 3C shows velocity index, rate of thrombin generation; FIG. 3D shows peak time, time to peak thrombin; and FIG. 3E shows lag phase, time to thrombin clot initiation. Note significant differences in children with AD compared to those without in total thrombin generation (AUC) and a trend towards a difference in peak thrombin and velocity index.

FIGS. 4A-4B are graphs showing the relationship between total thrombin generation and transepidermal water loss (TEWL) in subjects with atopic dermatitis (AD). FIG. 4A shows the association between total thrombin and TEWL at lesional skin sites. FIG. 4B shows the association between total thrombin and TEWL at never-lesional skin sites.

FIG. 5 shows that dabigatran significantly increases prothrombin time (PT) in mice (p-value<0.05) to approximately two-fold that of control mice.

FIGS. 6A-6D are graphs showing that thrombin inhibition using the direct oral thrombin inhibitor dabigatran attenuates AD development in a murine model. FIGS. 6A and 6B shows that thrombin inhibition results in decreased transepidermal water loss (TEWL) during disease development in mice. FIGS. 6C and 6D shows that thrombin inhibition results in decreased symptom severity scoring during disease development in a murine model.

FIGS. 7A-7B show that protease activated receptor 1 (PAR^{-/-}) deficiency does not attenuate AD development in a murine model. There was no difference in transepidermal water loss (TEWL) (FIG. 7A) or symptom scoring (FIG. 7B) between PAR^{-/-} mice or controls.

FIGS. 8A-8B are graphs showing mouse fibrin(ogen) skin staining and quantification in a murine model of AD. FIG. 8A shows immunohistochemistry (IHC) of skin samples from mice in experimental (Aspergillus (Asp) patch and Saline patch) and intervention (control chow and dabigatran chow). Note that fibrin(ogen) deposition is significantly lower in Asp mice that received dabigatran compared to controls. FIG. 8B shows fibrin(ogen) level quantification via enzyme-linked immunosorbent assay (ELISA) on skin sample homogenates from mice in each group.

FIGS. 9A-9B are graphs showing that complete (Fib^{-/-}) and partial (Fib^{+/-}) fibrinogen deficiency attenuates AD development in a murine model. FIG. 9A shows that fibrinogen deficiency results in decreased transepidermal water loss (TEWL) during disease development in mice. FIG. 9B shows that thrombin inhibition results in decreased symptom severity scoring during disease development in a murine model. Notably, both TEWL and symptom scoring in fibrinogen deficient mice was similar to controls.

FIG. 10 is a graph showing the assessment of absorption of topically applied bivalirudin (a direct thrombin inhibitor) by measuring prothrombin time.

### DETAILED DESCRIPTION OF THE INVENTION

Thrombin, also known as plasma thrombin, fibrinogenase, thrombase, thrombofort, topical, thrombin-C, tropostasin, activated blood-coagulation factor II, blood-coagulation factor IIa, etc., is an enzyme found in blood plasma and involved in the coagulation cascade. Prothrombin (coagulation factor II) is proteolytically cleaved to form thrombin in the clotting process. Thrombin in turn acts as a serine protease that converts soluble fibrinogen (also known as plasma fibrinogen) into insoluble strands of fibrin, as well as catalyzing many other coagulation-related reactions.

The present disclosure reports that children with atopic dermatitis (AD) have dysregulated clotting and increased thrombin generation, and that in preclinical studies using a model of AD in mice, thrombin inhibition through an exemplary direct thrombin inhibitor, dabigatran, increased barrier function and attenuated disease development and severity. In subsequent preclinical studies using a mouse model of AD, mice with both complete and partial fibrinogen deficiency showed increased barrier function and attenuated disease development and severity. Furthermore, topical application of another exemplary direct thrombin inhibitor, bivalirudin, was shown absorbed by skin and retains its function of inhibiting thrombin. The collective results reported herein suggest that thrombin and/or fibrinogen can serve as diagnostic biomarkers and treatment target for atopic dermatitis (AD).

Accordingly, the present disclosure provides methods for diagnosing atopic dermatitis (AD) by using thrombin and fibrinogen as biomarkers to determine the presence or severity of AD. Also provided are methods for treating AD by targeting thrombin.

### I. Method for Treating Atopic Dermatitis

In one aspect, the present disclosure provides a method for treating atopic dermatitis in a subject, the method comprising: administering to a subject in need of the treatment an effective amount of a composition comprising a therapeutic agent inhibiting plasma thrombin and a pharmaceutically acceptable carrier.

### A. Thrombin Inhibitors

In some aspects, provided herein are therapeutic agents that inhibits plasma thrombin for use in treating atopic dermatitis.

As used herein, a “therapeutic agent” refers to any substance (e.g., a small organic molecule, a nucleic acid, or a polypeptide) that has or expected to have a therapeutic beneficial effect on the health and well-being of a subject having or suspected of having a target disease such as AD as disclosed herein (e.g., eliminating or reducing the severity of AD) when administered in a therapeutically effective amount to the subject (e.g., a human patient). As used herein, a “therapeutic agent inhibiting plasma thrombin” refers to a molecule (e.g., a small organic molecule, a nucleic acid, or a polypeptide) or a combination of molecules that inhibits the formation of thrombin and/or inactivates thrombin, directly or indirectly, during clotting of plasma (e.g., plasma in skin blood vessels or capillaries), resulting in a reduced level of thrombin (e.g., total plasma thrombin and/or peak plasma thrombin) and/or a reduced level of thrombin activity.

In some examples, the therapeutic agent inhibiting plasma thrombin is a thrombin inhibitor, which can be a molecule that bind to and inhibit the activity of thrombin, or interferes with expression or degradation of thrombin so as to reduce its level, thereby suppressing or preventing blood clot formation.

The term “inhibiting” implies no specific mechanism of biological action whatsoever, and is deemed to expressly include and encompass all possible pharmacological, physiological, and biochemical interactions with thrombin, directly or indirectly. For purpose of the present disclosure, it will be explicitly understood that the term “inhibiting” encompass all the previously identified terms, titles, and functional states and characteristics whereby the thrombin itself (e.g., human thrombin), a thrombin bio-logical activity (including but not limited to its role in blood clotting), or the consequences of the biological activity, are substantially nullified, decreased, or neutralized in any meaningful degree, e.g., by at least 20%, 50%, 70%, 85%, 90%, 100%, 150%, 200%, 300%, or 500%, or by 10-fold, 20-fold, 50-fold, 100-fold, 1000-fold, or 104-fold.

Thrombin inhibitors can be categorized as indirect thrombin inhibitors or direct thrombin inhibitors. Examples of indirect thrombin inhibitors include heparin and warfarin. Direct thrombin inhibitors (DTIs) act as anticoagulants by directly binding to and inhibiting the enzymatic activity of thrombin. Non-limiting examples of DTI include dabigatran, bivalirudin, argatroban, hirudin, lepirudin, desirudin, argatroban, inogatran, melagatran, and ximelagatran. In some embodiments, the therapeutic agent inhibiting plasma thrombin is a direct thrombin inhibitor. In some embodiments, the therapeutic agent inhibiting plasma thrombin is dabigatran, bivalirudin, argatroban, hirudin, lepirudin, desirudin, argatroban, inogatran, melagatran, or ximelagatran.

Additional agents that inhibit thrombin may include antibodies neutralizing the activity of thrombin. Such neutralizing antibodies can be prepared via routine practice. In addition, the agent that inhibits thrombin may be a nucleic acid-based molecule, for example, an anti-sense nucleic acid directed to a nucleic acid encoding thrombin, a small interfering RNA (siRNA) that targets the messenger RNA encoding thrombin, or a microRNA that regulates expression of thrombin.

It is routine to prepare antisense oligonucleotide molecules that specifically bind a target mRNA without cross-reacting with other polynucleotides. Exemplary sites of targeting include, but are not limited to, the initiation codon, the 5′ regulatory regions, the coding sequence and the 3′ untranslated region. In some embodiments, the oligonucleotides are about 10 to 100 nucleotides in length, about 15 to 50 nucleotides in length, about 18 to 25 nucleotides in length, or more. The oligonucleotides can comprise backbone modifi-cations such as, for example, phosphorothioate linkages, and 2′-0 sugar modifications well known in the art. Alternatively, thrombin expression and/or release can be decreased using gene knockdown, morpholino oligonucleotides, small interfering RNA (siRNA or RNAi), microRNA or ribozymes.

Any of the therapeutic agents that inhibit thrombin as disclosed herein may be used in the method for treating AD as also disclosed herein.

### B. Pharmaceutical Compositions

One or more of the above-described thrombin inhibitors can be mixed with a pharmaceutically acceptable carrier (e.g., excipient, buffer) to form a pharmaceutical composition for use in treating AD.

A “pharmaceutically acceptable carrier” as used herein means a pharmaceutically acceptable material, composition, or vehicle, such as a liquid or solid filler, diluent, excipient, solvent, or encapsulating material. Each carrier must be “acceptable” in the sense of being compatible with the active ingredient of the composition (and preferably, capable of stabilizing the active ingredient) and not deleterious to the subject to be treated. Pharmaceutically acceptable carriers are well known in the art and include, for example, aqueous solutions such as water or physiologically buffered saline or other solvents or vehicles such as glycols, glycerol, oils, or injectable organic esters. Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG). The choice of a pharmaceutically acceptable carrier, including a physiologically acceptable agent, depends, for example, on the route of administration of the composition. Further details of pharmaceutically acceptable carriers may be found in e.g., Remington: The Science and Practice of Pharmacy 20th Ed. (2000) Lippincott Williams and Wilkins, Ed. K. E. Hoover.

In some examples, the pharmaceutical composition described herein is a topical formulation containing surfactants, stearic acid, thickener (e.g., carbomer) and preservatives. In some examples, the pharmaceutical composition described herein comprises liposomes containing the thrombin inhibitor, which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The thrombin inhibitor-containing pharmaceutical composition may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

In other examples, the pharmaceutical composition described herein can be formulated in sustained-release format. Suitable examples of sustained-release preparations include semipermeable matrices of solid hydrophobic polymers containing the thrombin inhibitor, which matrices are in the form of shaped articles, e.g., films, or microcapsules. Examples of sustained-release matrices include polyesters, hydrogels (for example, poly(2-hydroxyethyl-methacrylate), or poly(v nylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic thrombin inhibitor compositions may be placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid pre-formulation composition containing a homogeneous mixture of a compound of the present invention, or a non-toxic pharmaceutically acceptable salt thereof. When referring to these pre-formulation compositions as homogeneous, it is meant that the active ingredient is dispersed evenly throughout the composition so that the composition may be readily subdivided into equally effective unit dosage forms such as tablets, pills and capsules. This solid pre-formulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%.

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect. Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

In some embodiments, the pharmaceutical composition comprising the thrombin inhibitor described herein may be an aqueous formulation, which may further comprise a buffer (which may comprise an amino acid such as histidine or arginine), a salt (e.g., sodium chloride), and/or a surfactant, such as a nonionic surfactant. Such an aqueous formulation may be used in any of the method disclosed herein for topical administration.

In some examples, provided herein is a lyophilized formulation for bivalirudin, which can be lyophilized from an aqueous solution comprising about 40-60 mg/ml bivalirudin, about 20-30 mg/ml mannitol, and a pH of about 5-6. In some examples, the aqueous solution comprises 50 mg/ml of bivalirudin and 25 mg/ml mannitol. In some examples, the aqueous solution comprises NaOH for pH adjustment. The lyophilized formulation may be reconstituted with water or a buffering agent to form an aqueous formulation comprising about 40-60 mg/ml bivalirudin (e.g., 50 mg/ml), about 20-30 mg/ml mannitol (e.g., 25 mg/ml), and having a pH of about 7.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

### C. Use of Thrombin Inhibitors for Treating Atopic Dermatitis

To practice the method disclosed herein, an effective amount of the pharmaceutical composition described above can be administered to a subject (e.g., a human) in need of the treatment.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having AD. A subject having an AD by routine medical examination, e.g., examining patient's family history and visual assessment of the skin (e.g., by a scoring system), and/or using the diagnosis method for AD disclosed herein, as discussed below. In some embodiments, the subject is a human adult patient having atopic dermatitis. In some embodiments, the subject is a human pediatric patient having atopic dermatitis.

A subject suspected of having AD might show one or more symptoms of the disorder, e.g., dry, cracked skin; itchiness (pruritus); rash on swollen skin that varies in color depending on your skin color; small, raised bumps, on brown or black skin; oozing and crusting; thickened skin; darkening of the skin around the eyes; raw, sensitive skin from scratching.

For assessing the severity of AD, various scoring scales may be used, such as Eczema Area and Severity Index (EASI), Severity Scoring of Atopic Dermatitis (SCORAD), patient-oriented SCORAD (PO-SCORAD), and Patient-Oriented Eczema Measure (POEM). In some embodiments, the subject is diagnosed as having moderate-to-severe atopic dermatitis using a SCORing Atopic Dermatitis (SCORAD) index scoring system. In some embodiments, the subject is diagnosed as having moderate-to-severe atopic dermatitis using an Eczema Area and Severity Index (EASI) scoring system. In some embodiments, the subject has moderate atopic dermatitis. In some embodiments, the subject has severe atopic dermatitis. In some embodiments, the subject has moderate-to-severe atopic dermatitis.

An “effective amount” as used herein refers to the amount of each active agent required to confer therapeutic effect on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of AD. Alternatively, sustained continuous release formulations of a thrombin inhibitor may be appropriate. Various formulations and devices for achieving sustained release are known in the art.

For the purpose of the present disclosure, the appropriate dosage of a thrombin inhibitor will depend on the specific thrombin inhibitor(s) (or compositions thereof) employed, the type and severity of AD, whether the thrombin inhibitor is administered for preventive or therapeutic purposes, previous therapy, the patient's clinical history and response to the thrombin inhibitor, and the discretion of the attending physician. Typically, the clinician will administer a thrombin inhibitor, until a dosage is reached that achieves the desired result. Administration of a thrombin inhibitor can be continuous or intermittent, depending, for example, upon the recipient's physiological condition, whether the purpose of the administration is therapeutic or prophylactic, and other factors known to skilled practitioners.

In some embodiments, the therapeutic agent may reduce the level of thrombin (e.g., total plasma thrombin and/or peak plasma thrombin) in the subject receiving such by at least 20%, e.g., at least 30%, 40%, 50%, 60%, 70%, or above, as compared with the thrombin level in the subject prior to the treatment.

As used herein, the term “treating” refers to the application or administration of a composition including one or more active agents to a subject, who has AD, a symptom of AD, or a predisposition toward the disease, with the purpose to cure, heal, alleviate, relieve, alter, remedy, ameliorate, improve, or affect the disorder, the symptom of the disease, or the predisposition toward the disease.

Alleviating a disease associated with AD includes delaying the development or progression of the disease, or reducing disease severity. Alleviating the disease does not necessarily require curative results. As used therein, “delaying” the development of a disease (such as AD) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease associated with AD includes initial onset and/or recurrence.

Also within the scope of the present disclosure are preventive treatments of AD with any of the thrombin inhibitor to reduce the risk for occurrence of such a disorder. Subjects suitable for such a preventive treatment may be human patients having history of AD and/or family history of AD.

Various administration methods, known to those of ordinary skill in the art of medicine, can be used to administer the pharmaceutical composition to the subject, depending upon factors such as the severity of AD to be treated or the site of the AD. This composition can be administered via a number of administration routes, e.g., administered orally, parenterally, by inhalation spray, topically, rectally, nasally, buccally, vaginally or via an implanted reservoir.

The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble thrombin inhibitors can be administered by the drip method, whereby a pharmaceutical formulation containing the thrombin inhibitor and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the thrombin inhibitor, can be dissolved and administered in a pharmaceutical excipient such as Water-for-Injection, 0.9% saline, or 5% glucose solution.

In some embodiments, a thrombin inhibitor as described herein, for example, dabigatran or bivalirudin, is used for treating atopic dermatitis as follows. Atopic dermatitis, also known as eczema, is a chronical skin condition characterized by redness and/or itchy. It is common in children but can occur at any age. A patient who needs the treatment can be identified by routine medical practice as having one or more symptoms of atopic dermatitis, including dry skin, itching, red to brownish-gray patches, small, raised bumps, which may leak fluid and crust over when scratched, thickened, cracked, scaly skin, and/or raw, sensitive, swollen skin from scratching. In some instances, the severity of AD of a candidate subject can be examined via routine practice. If the AD severity score of the candidate subject (e.g., by SCORAD or EASI scoring system) is higher than a normal level (representing the average AD severity in subjects of the same species, e.g., humans, who are free of atopic dermatitis or have only mild AD), the subject is administered an effective amount of a topical formulation containing a thrombin inhibitor such as dabigatran or bivalirudin and a pharmaceutically acceptable carrier such as an oil-in-water emulsion containing surfactants, stearic acid, thickener (e.g., carbomer), and preservatives.

In some embodiments, the subject has an increased level of total plasma thrombin or peak plasma thrombin as compared to a reference value for total plasma thrombin or peak plasma thrombin. See disclosures below.

Fibrinogen is a major thrombin substrate that gets converted into fibrin during clotting. In some embodiments, the subject has an increased level of plasma fibrinogen as compared to a reference value for plasma fibrinogen. In some embodiments, the level of plasma fibrinogen is measured by an immunohistochemistry (IHC) staining assay or an enzyme-linked immunosorbent assay (ELISA).

In some embodiments, the subject has a skin barrier dysfunction. Skin barrier dysfunction may be measured by the level of transepidermal water loss (TEWL). In some embodiments, the subject has an increased level of transepidermal water loss (TEWL) as compared to a reference value for TEWL. TEWL may be measured in various applicable methods and instruments. In some embodiments, the level of transepidermal water loss (TEWL) is measured using a Dermalab instrument.

Carriers of risk alleles for common prothrombotic genetic polymorphisms such as Factor V Leiden (FFL, rs6025) or Prothrombin G20210A (rs1799963) may have increased thrombin generation potential and an increased risk for thromboembolic events. In some embodiments, the subject is free of a genetic thrombophilic disorder associated with a Factor V Leiden (FFL, rs6025) mutation or a Prothrombin G20210A (rs1799963) mutation.

In some embodiments, the therapeutic agent is a direct thrombin inhibitor. As used herein, a “direct thrombin inhibitor” refers to a therapeutic agent that binds to and directly inhibits enzymatic activities of thrombin without requiring a cofactor, such as antithrombin, to exert its inhibiting effect. In some embodiments, the direct thrombin inhibitor comprises dabigatran, bivalirudin, argatroban, hirudin, lepirudin, desirudin, argatroban, inogatran, melagatran, ximelagatran, or a combination thereof.

Methods disclosed herein may be used together with various administration routes. The therapeutic agent inhibiting plasma thrombin can be administered to the subject either locally or systemically. In some embodiments, the composition is administered orally, parenterally, intravenously, and topically.

Moreover, the present disclosure provides a lyophilized formulation, which is lyophilized from an aqueous solution comprising about 40-60 mg/ml bivalirudin, about 20-30 mg/ml mannitol, and a pH of about 5-6. In some embodiments of the lyophilized formulation, the aqueous solution comprises 50 mg/ml of bivalirudin and 25 mg/ml mannitol. In some embodiments of the lyophilized formulation, the aqueous solution comprises NaOH for pH adjustment.

Further provided herein is a method for treating a skin disorder in a subject, the method comprising: (i) providing the lyophilized formulation of any preceding embodiments, (ii) reconstituting the lyophilized formulation with water or a buffering agent to produce a final aqueous formulation comprising about 40-60 mg/ml bivalirudin, about 20-30 mg/ml mannitol, and having a pH of about 7; and (iii) administering the final aqueous formulation topically to the subject. In some embodiments, the final aqueous formulation comprises 50 mg/ml bivalirudin and 25 mg/ml mannitol, and has a pH of about 7. In some embodiments, the subject is a human patient having atopic dermatitis; optionally wherein the human patient is a pediatric patient.

### II. Method for Diagnosing Atopic Dermatitis

In another aspect, the present disclosure provides a method for diagnosing atopic dermatitis in a subject, the method comprising: (a) measuring a level of a biomarker in one or more biological samples from a subject suspected of having atopic dermatitis; (b) comparing the level of the biomarker in the biological sample(s) with a reference value for the biomarker; and (c) determining presence or severity of atopic dermatitis in the subject based on the comparing results of (b), wherein an elevated level of the biomarker in the biological sample(s) relative to the reference value is indicative of presence or has moderate-to-severe atopic dermatitis in the subject; wherein the biomarker is thrombin, fibrinogen, transepidermal water loss (TEWL), or a combination thereof.

### A. Biomarkers for Atopic Dermatitis

Some aspects of the present disclosure relate to methods for diagnosing atopic dermatitis using one or more biomarkers.

As used herein, the terms “diagnose”, “diagnosing” and “diagnostic” refer to the process of determining a disease state or disorder in a subject. In determining disease state, a clinician might classify one or more characteristics of a subject, such as, for example, symptoms and/or biomarkers.

As used herein, a “biomarker” refers to a distinctive biological or biologically derived indicator of a process, event, or condition. In some examples, the biomarker is a molecule whose measurement or level provides information regarding the state or a condition of a subject, e.g., the presence or severity of atopic dermatitis. In certain embodiments, the biomarker is a gene or gene product (e.g., a transcript or a polypeptide). In some instances, the biomarker is a plurality of molecules or indicators. In some other instances, the biomarker is a composite index or calculated value derived from a plurality of genes, gene products, and/or biological processes.

In some embodiments of the present disclosure, the biomarker comprises total plasma thrombin, peak plasma thrombin, thrombin velocity index, thrombin peak time, thrombin lag phase, or a combination thereof. In some embodiments, the biomarker further comprises plasma fibrinogen, and/or TEWL. Such biomarkers can be used to diagnose the presence or severity of atopic dermatitis, as further described below.

### B. Biological Samples and Assays

Some aspects of the method relate to measuring the level of the biomarker in one or more biological samples from a subject in order to diagnose AD.

As used herein, a “biological sample” refers to a sample of biological material obtained from a subject, including sample of biological tissue or fluid origin obtained in vivo or in vitro. Such samples can be, but are not limited to, bodily fluid (e.g., blood, blood plasma, serum, or urine), organs, tissues, fractions, and cells isolated from mammals including, humans. Biological samples also may include sections of the biological sample including tissues (e.g., sectional portions of an organ or tissue). Biological samples may also include extracts from a biological sample, for example, an antigen from a biological fluid (e.g., blood or urine). In some examples, the one or more biological samples comprise a blood sample, a skin sample, or a combination thereof.

The biomarkers of the present disclosure may be measured in any suitable biological samples. For instance, the biomarker is total plasma thrombin, peak plasm thrombin, and/or fibrinogen, and the level of total plasma thrombin, peak plasm thrombin, and/or fibrinogen is measured in a blood sample of the subject. In another instance, the biomarker is TEWL, and the level of TEWL is measured in a skin sample of the subject.

Based on the type of biomarker and biological sample, a suitable assay is used to measure a level of the biomarker in the biological sample. Methods and techniques of the suitable assays are known in the art. In some instances, the level of total plasma thrombin or peak plasma thrombin is measured by a thrombin generation assay (TGA). In some embodiments, the TGA is the Technothrombin® Thrombin Generation Assay.

A thrombin generation assay (TGA) or thrombin generation test (TGT) is a global coagulation assay (GCA) and type of coagulation test that determines the rate and extent of thrombin in a patient sample (e.g., plasma sample), which can be used to assess coagulation and thrombotic risk. It is based on the potential of a plasma to generate thrombin over time, following activation of coagulation via addition of phospholipids, tissue factor, and calcium. The results of the TGA can be output as a thrombogram or thrombin generation curve using computer software with calculation of thrombogram parameters.

The main thrombogram parameters for the TGA include:
1) Total thrombin, also known as total plasma thrombin, total thrombin generation, endogenous thrombin potential (ETP), or area under the curve (AUC) of the thrombin generation curve which measures the level of thrombin generated in the sample in e.g., nanomoles;
2) Peak thrombin, also known as peak plasma thrombin, peak or maximum concentration of thrombin generated, in unit of molar concentration (e.g., nM);
3) Velocity index, also known as thrombin velocity index, rate of thrombin generation, slope of thrombin generation between lag time/first thrombin generation and time to peak; corresponds to first derivative of this part of curve;
4) Peak time, also known as thrombin peak time, time to peak or ttPeak, time to maximum concentration of thrombin generated, in unit of e.g., minutes; and
5) Lag phase, also known as thrombin lag phase, thrombin lag time, lag time, time until thrombin first generated/thrombin concentration first increased, in unit of e.g., minutes;

Additional thrombogram parameters for the TGA may also include start tail (time at which thrombin generation ends and all generated thrombin has been inhibited, in unit of e.g., minutes). Further details of the TGA can be referred to, e.g., Salvagno, Gian Luca, and Erik Berntorp. “Thrombin generation assays (TGAs).” Hemostasis and Thrombosis. Humana Press, New York, NY, 2017. 515-522.

In some instances, the level of the fibrinogen is measured by an immunohistochemistry (IHC) staining assay or an enzyme-linked immunosorbent assay (ELISA). Methods of protocols of IHC and ELISA may be referred to, e.g., Key, Marc. “Immunohistochemistry staining methods.” Education Guide Immunohistochemical Staining Methods Fourth Edition (2006): 47 and Crowther, John R. The ELISA guidebook. Vol. 566. New York, NY, USA: Humana press, 2009, respectively.

In certain embodiments of the present disclosure, the subject has a skin barrier dysfunction, which can be measured by the level of transepidermal water loss (TEWL). TEWL may be measured in various applicable methods and instruments, including, e.g., a Dermalab instrument. See, e.g., Grove, Gary L., et al. “Computerized evaporimetry using the DermaLab® TEWL probe.” Skin Research and Technology 5.1 (1999): 9-13.

### C. Determination of Atopic Dermatitis Presence or Severity

Some aspects of the method relate to comparing the level of the biomarker in the one or more biological samples with a reference value for the biomarker and making a determination of presence or severity of atopic dermatitis based on the comparison.

As used herein, a “reference value” refers to the level of the corresponding biomarker (e.g., thrombin such as total plasma thrombin and/or peak plasma thrombin or plasma fibrinogen as disclosed herein) measured in a control sample. The reference value may be measured using the same method for measuring the level of the same biomarker in a biological sample from a candidate subject. The control sample may be of a control subject or a population of control subjects.

In some embodiments, the control subject or population of control subjects may be a subject or subjects who do not exhibit signs or symptoms of atopic dermatitis. A reference value obtained from such a control subject(s) may be representative of the level of the corresponding biomarker (e.g., those disclosed herein) in healthy subjects (subjects who are free of atopic dermatitis). The level of the biomarker from a candidate subject being higher than such a reference value would be indicative of presence of atopic dermatitis in the candidate subject.

In some embodiments, the control subject or population of control subjects may be a subject or subjects who have mild atopic dermatitis as diagnosed by routine medical practice. A reference value obtained from such a control subject(s) may be representative of the level of the corresponding biomarker (e.g., those disclosed herein) in mild atopic dermatitis. The level of the biomarker from a candidate subject being higher than such a reference value would be indicative of moderate-to-severe atopic dermatitis in the candidate subject.

It is to be understood that the methods provided herein do not require that the reference value be measured every time a subject is tested. Rather, in some embodiments, it is contemplated that the reference value can be obtained and recorded from a suitable control subject(s) and any test level may be compared with such a pre-determined value. In some instances, the reference value may be a single cutoff value. In other instances, the reference value can be a range of values.

Accordingly, when the level of the biomarker, such as total plasma thrombin, peak plasma thrombin, thrombin velocity index, fibrinogen, and/or TEWL, measured in the subject's biological sample(s) is elevated relative to the reference value of a control subject or population of control subjects having no AD, a determination is made that the subject has AD. In other instances, when the level of the biomarker, such as total plasma thrombin, peak plasma thrombin, thrombin velocity index, fibrinogen, and/or TEWL, measured in the subject's biological sample(s) is elevated relative to the reference value of a control subject or population of control subjects having mild AD, a determination is made that the subject has moderate-to-severe AD.

In other examples, when the level of the biomarker, such as thrombin peak time and/or thrombin lag phase, measured in the subject's biological sample(s) is decreased relative to the reference value of a control subject or population of control subjects having no AD, a determination is made that the subject has AD. In other instances, when the level of the biomarker, such as thrombin peak time and/or thrombin lag phase, measured in the subject's biological sample(s) is decreased relative to the reference value of a control subject or population of control subjects having mild AD, a determination is made that the subject has moderate-to-severe AD.

The diagnosis methods disclosed herein may be used on any applicable subjects. In some examples, the subject is a human adult or pediatric patient having atopic dermatitis. In some examples, the subject is free of a genetic thrombophilic disorder associated with a Factor V Leiden (FFL, rs6025) mutation or a Prothrombin G20210A (rs1799963) mutation.

D. Treatment after Diagnosis

In some aspects, the method further comprises treating the subject who is determined as having atopic dermatitis in step (c) a therapeutic agent inhibiting plasma thrombin. See disclosures above for such treatment aspects.

### III. Kits for Use in Diagnosing or Treating Atopic Dermatitis

The present disclosure also provides kits for use in diagnosing or treating AD in a subject. Such kits can include one or more containers comprising a thrombin inhibitor as described herein.

In some embodiments, the kit can comprise instructions for use in accordance with any of the methods described herein. The included instructions can comprise a description of administration of the thrombin inhibitor to treat, delay the onset, or AD according to any of the methods described herein. The kit may further comprise a description of selecting an individual suitable for treatment based on identifying whether that individual has, is suspected of having, or is at risk for the disorder. In still other embodiments, the instructions comprise a description of administering a thrombin inhibitor to a subject in need of the treatment to reduce the risk for developing AD.

The instructions relating to the use of a thrombin inhibitor generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating AD. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a thrombin inhibitor, such as dabigatran or bivalirudin.

Kits may optionally provide additional components such as buffers and interpretive information. Normally, the kit comprises a container and a label or package insert(s) on or associated with the container. In some embodiments, the present disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984; Animal Cell Culture (R. I. Freshney, ed. (1986»; Immobilized Cells and Enzymes (IRL Press, (1986; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1. Thrombin and Fibrinogen Contribute to Atopic Dermatitis Pathogenesis

### Introduction

Atopic dermatitis (AD) affects approximately 1 in 10 children in the United States and is increasing in incidence. Nearly 80% of children with AD go on to develop other allergic disorders, highlighting its importance within the spectrum of allergic diseases. In addition to increasing risk for subsequent allergic disease development, AD is associated with an increased risk for other disorders including cardiovascular events. Numerous studies have shown that adults with AD have a higher risk for thromboembolic events and adverse cardiac outcomes compared to adults without AD. Further, this increased risk is severity dependent, whereby adults with severe AD have a higher risk for thromboembolic events compared to adults with mild or moderate AD. In 2010, Nastalek et al. found that adults with atopy had longer clot lysis times compared with non-atopic adults (Nastalek et al., J Thromb Thrombolysis, 2010). In addition, Undas et al found that, when compared with adults without AD, adults with AD had lower clot permeability coefficients, longer clot lag phases, and longer fibrinolysis times (Undas et al., J Thromb Haemost, 2011). Together, these studies suggest that AD represents a thrombophilic state. However, these studies were done in adult populations and there is currently no data available regarding whether children with AD have altered hemostatic function. Moreover, there are no studies investigating the mechanistic relationship between AD and clotting to explain these associations in any age group.

This represents a significant gap in the current literature since children represent the group with the highest AD prevalence and AD disease-related burden. In addition, elucidation of the impact of clotting on AD has significant implications for disease management. To determine whether AD represents a thrombophilic state in children, the Mechanisms of Progression of Atopic Dermatitis to Asthma in Children (MPAACH) cohort, the first US early life cohort of children with AD, were used to determine if AD severity, barrier dysfunction, and/or allergic sensitization are associated with altered hemostatic system regulation in early childhood. A mouse model of AD was also established to determine the mechanistic relationship between clotting and AD pathogenesis.

### Materials and Methods

### MPAACH Study Design

MPAACH subjects were identified from the electronic medical record at Cincinnati Children's Hospital Medical Center (CCHMC) or from the public through direct advertising. Children were 4 months-2 years of age at enrollment and were previously full-term (gestation ≥36 weeks). All children had an AD diagnosis using either Hanifin and Rajka criteria (Hanifin and Rajka, Acta Derm Venereol, 1980) or parent/legal guardian report of AD with the Children's Eczema Questionnaire (von Kobyletzki et al., Dermatology, 2013). MPAACH exclusion criteria included: immunosuppression, co-morbid lung disease, bleeding diathesis, or inability to donate biologic samples. The participant's legally authorized representative (LAR)(s) provided parental permission for participation in MPAACH. The MPAACH study is approved by the Cincinnati Children's Hospital Medical Center Institutional Review Board. Parent(s)/LAR(s) of MPAACH subjects answered questionnaires at their visit related to clinical (asthma, allergic rhinitis, food allergy, and atopic dermatitis), demographic, environmental, and family histories. Each child underwent food (milk, egg, peanut, wheat, soy) and aero-allergen (trees (tree mix 5 (Maple, Oak, Pecan, Sycamore and Willow (Black)) and tree mix 6 (White Ash, Birch Mix (Red and White), Black Walnut, Common Cottonwood and American Elm), ragweed, weeds, mold, grass, cat, dog, cockroach, dust mite) evaluation via skin prick testing (SPT), trans-epidermal water loss (TEWL) measurement, and SCORAD assessment at their first MPAACH visit (Biagini Myers et al., J Allergy Clin Immunol Pract, 2020). SCORAD severity was defined by total: mild (<25), moderate (25-49), and severe (>50). Platelet counts were determined as part of a complete blood count performed on MPAACH participants who had sufficient biological samples.

### Analyses of Hemostatic Function

All subjects underwent clotting analyses using banked plasma samples. For all clotting assays, plasma from sodium EDTA tubes was transferred into conical tubes containing Ficoll-Paque Premium Sterile solution (Ref. 17-5442-02) and spun at 2200 RPM at room temperature to allow for PBMC isolation. Plasma was collected, aliquoted, and stored at −80 C. All MPAACH blood samples were processed within 4 hours of collection and kept on a rocker until processed.

### Thrombin Generation Assay:

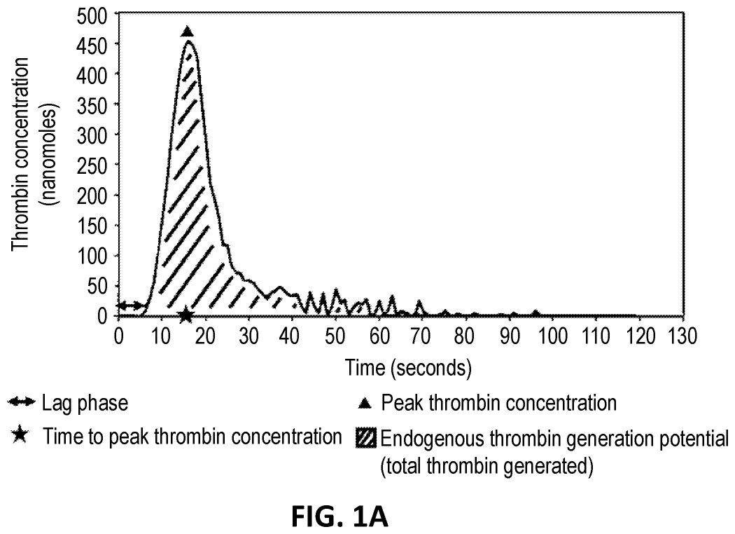

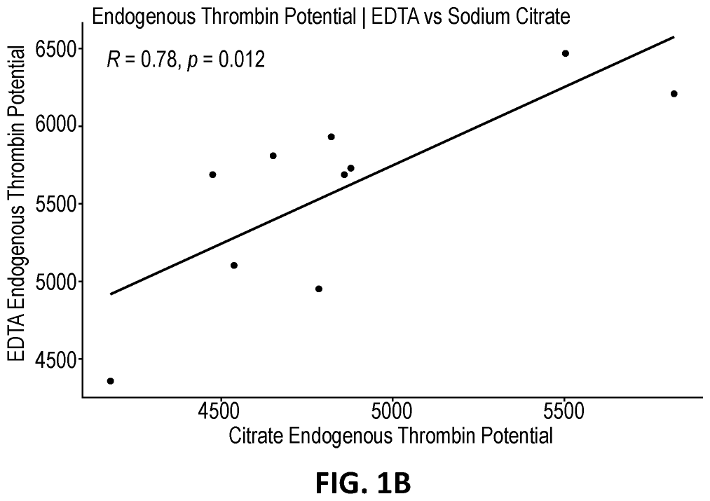

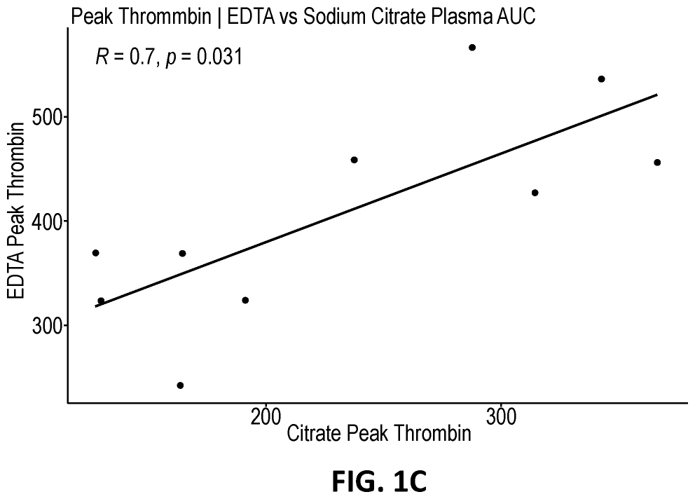

The Technothrombin® Thrombin Generation Assay (TGA, Catalog #: 5006010) was used to assess thrombin generation potential in human plasma samples. The TGA introduces micelles containing recombinant human tissue factor (1 pM) to human blood plasma to stimulate the extrinsic coagulation cascade and analyze thrombin generation potential. Activated thrombin cleaves the TGA substrate and creates detectable fluorescence. The TGA creates a thrombin generation curve (TGC) for each sample. The TGC determines the lag time prior to initial thrombin formation, peak thrombin, time to peak thrombin, the velocity index of the TGC, and the area under the TGC, which serves as a proxy for total thrombin generation potential. For all plasma samples, the TGA calibration kit was used to set the parameters for TGC formation of subsequent assays. The BioTek Synergy H1 Hybrid Reader was warmed to 37° C. Lyophilized reagents were resuspended and kept at room temperature. Frozen plasma was warmed to room temperature. Each assay was run alongside Technothrombin-provided positive controls with either increased or decreased thrombin generation (Cat. 5006320. Cat. 5006330, respectively). Plasma and positive controls were added to a black NUNC Maxisorp 96-well plate (ThermoFisherScientific Ref. 475515). Reagent C Low (1 pM final, Cat. 5006212) was added to all samples and controls. The reaction was initiated once Technothrombin TGA Substrate (Cat. 5006235) was added to the samples, therefore TGA substrate was added last. The reaction was introduced to the plate reader immediately after the addition of TGA substrate. The BioTek Synergy H1 Hybrid Reader was used to take end-point fluorescence readings every minute for 2 hours with an excitation filter set to 360/40 nm, emission filter set to 460/40 nm, and optics position set to top 400 nm. The Technothrombin TGA Evaluation Software then created a thrombin generation curve (FIG. 1A) TGC for each sample. The correlation of key TGA parameters was evaluated in subjects that had both EDTA and citrate samples. There was a strong correlation between sample types for total thrombin generated (r=0.78, P-value=0.0012, FIG. 1B) and peak thrombin (r=0.7, P-value=0.031, FIG. 1C).

### Factor V Leiden Genotype:

MPAACH subjects underwent genotypic analysis using the Infinium Multi-Ethnic Global-8 v1.0 kit (Illumina), which includes data on the risk variant at rs6025 for Factor V Leiden (FFL). All samples were processed and analyzed as per the standard Illumina protocol with no modifications. General population allele frequencies for rs6025 were obtained from dbSNP (Sherry et al., Nucleic Acids Res, 2001).

### Prothombin G20210a Genotype:

The prothrombin G20210A mutation genotype at rs1799963 was assessed in MPAACH subjects using the Taqman Genotyping Assay (Thermo Fisher Scientific, Catalog: 4351379, Assay ID: C 8726802_20). DNA was extracted from either the blood or saliva of subjects. Blood DNA was collected using the DNeasy Blood and Tissue kit (Qiagen, Catalog: 69504) and saliva DNA was obtained utilizing the ORAgene Discover kit (ID: OGR-675). In an UV-sterilized hood, Taqman ProAmp Master Mix (Catalog: A30866) was combined with the Taqman genotyping assay and nuclease-free water with a ratio of 20:1:17, respectfully, to create a working genotyping master mix that accounted for the concentrations of the reagents. Participant genomic DNA and working genotyping master mix were introduced into a 96-well plate with a ratio of 1:20, respectfully. Participants with known genotypes were included to confirm the validity of the genotyping assay during each run and were used as positive controls. Wells with either only Taq ProAmp Master Mix or nuclease-free water were used as negative controls. The microplate was immediately read using the Applied Biosystems Quant Studio 3 (Thermo Fisher Scientific, SN: 272311008). The cycle threshold (CT) values for both possible sequences involved in the G20210A polymorphism were assayed by Thermo Fisher Cloud Data Analysis Genotyping app to determine subject genotype. General population allele frequencies for G20210a were obtained from dbSNP (Sherry et al,).

### Mouse Models of AD Phenotype:

4- to 6-week-old mice were used for an established AD model in which the mice develop an AD-like phenotype following 3 week-long cutaneous allergen exposures with a 24-hour rest periods between subsequent Aspergillus fumigates (Asp) patch placements (Brandt et al.; Zhang et al.; Stevens et al.). After 3 patches mice were sacrificed using standard procedure and biospecimens were collected. All mouse studies were approved by CCHMC IACUC prior to initiation. Primary outcomes for the model were TEWL and symptom score after the third patch. TEWL was measured as previously described using methods established within the laboratory using methods established in the laboratory (Gupta et al., J Allergy Clin Immunol, 2008). Briefly, for TEWL measurements assess the change in water vapor density on the skin using a cutaneous probe. Measurements are recorded as grams per meter squared per hour after the rate of TEWL stabilizes and when the SD became 0.2 or less. AD severity was determined a standardized scoring assessment tool which includes visual inspection of skin redness, thickness, and excoriations based on the Eczema Area and Severity Index (EASI) scoring system (Brandt et al.; Zhang et al.; Stevens et al.; DaSilva-Arnold et al., Arch Dermatol Res, 2018).

Thrombin Inhibition with Dabigatran, Fibrinogen and PAR-1 Deficient Mice:

Mice received the direct thrombin inhibitor, dabigatran, incorporated into a standard chow mixture by Dyets, Inc. (Bethlehem, PA) at a dose of 7.5 mg/g of chow. A control chow without dabigatra using the same chow base was also obtained from Dyets. IncSkin and plasma fibrinogen levels were also evaluated using a fibrinogen ELISA (abCAM; ab208036). C57BL/6-derived fibrinogen-deficient (Fib−/−) and PAR-1 deficient (PAR-1−/−) mice have been previously described (Posma et al., Arterioscler Thromb Vasc Biol, 2019; Connolly et al., Am J Pathol, 1997).

### Statistical Methods

Frequency (percentage) and median (interquartile range) were used to characterize the study population. Simple logistic regression (for binary outcomes) and linear regression (for continuous outcomes) were used to test the associations between thrombin generation assay parameters and atopic dermatitis-related outcomes. The continuous atopic dermatitis-related outcomes were log transformed to meet the normality assumption. Boxplots and scatterplots were plotted to help visualize the correlations. For the murine models of AD TEWL was the primary outcome. Symptom scoring was a secondary outcome. To determine differences between murine groups in the dabigatran and fibrinogen AD models Wilcoxon tests were performed, with the primary comparison between patches 1 to 3 in the experimental model. For each phenotype of thrombin generation assay, the values obtained by EDTA anticoagulant were regressed on the values obtained by citrate anticoagulant and the regression models was applied to the non-AD controls with citrate anticoagulant to obtain the corrected ETDA values. Wilcoxon rank sum tests were performed to access the difference between non-AD controls and AD samples. Kruskal-Wallis rank sum tests were performed to test overall difference among non-AD controls, Mild ADs and Moderate/Severe ADs, followed by Wilcoxon tests for multiple pairwise comparisons. A nominal P-value threshold (p<0.05) was applied for significance. All the analyses were performed in R software, version 3.22.

### Results

### Subject Demographics

Plasma from a total of 81 children from the MPAACH cohort. Subjects with available plasma that had not undergone multiple freeze-thaw cycles were included in the analysis. Subject demographics are presented in Table 1. Except for age, overall, the subset of subjects was similar to the general MPAACH cohort.

TABLE 1
Subject demographics

| | Study Cohort | MPAACH* | |
|---|---|---|---|
| Demographic | (N = 81) | (N = 611) | P-value |
| Female n (%) | 37 (45.7%) | 292 (47.8%) | 0.81 |
| Black n (%) | 47 (58.0%) | 418 (68.5%) | 0.08 |
| Public insurance n (%) | 51 (63.0%) | 433 (70.9%) | 0.18 |
| Age, median (range) | 2.25 (0.45, 2.97) | 1.79 (0.27, 2.97) | 0.0005 |

*Excludes subjects included in the Study Cohort

Increased Plasma Thrombin Generation is Associated with AD Severity:

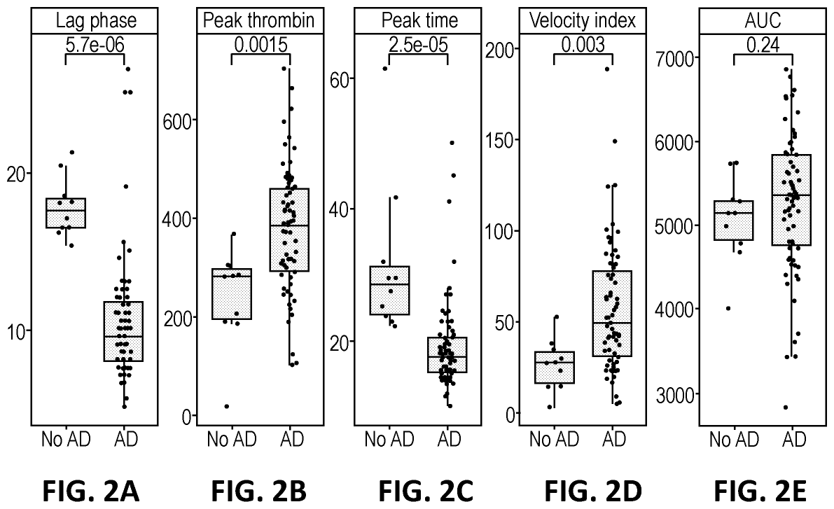

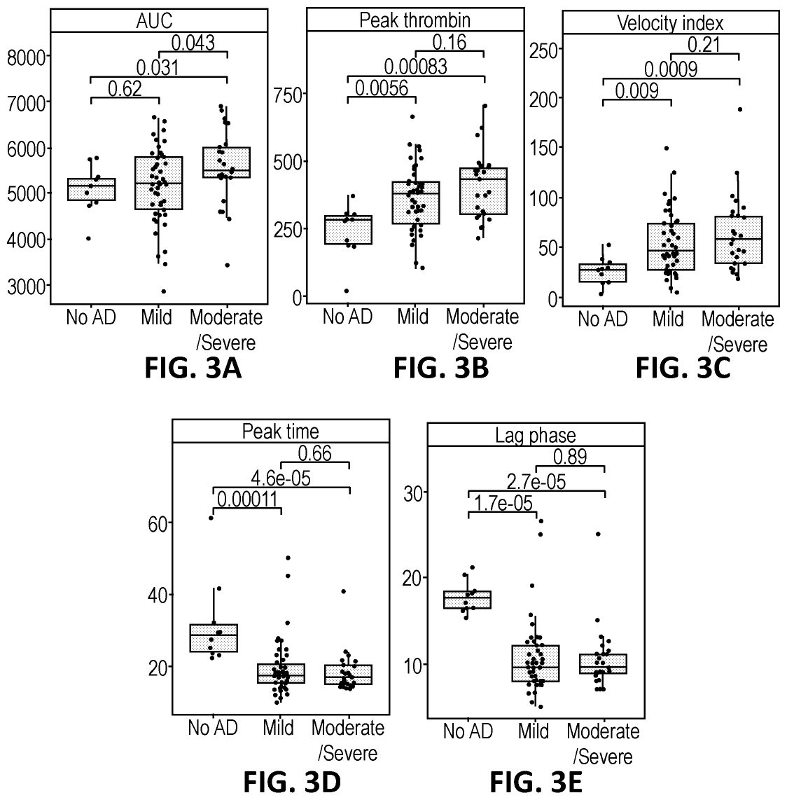

First assessed was if TGA parameters were impacted by AD in children compared with non-AD controls. Compared with controls, children with AD had a shorter time to clot initiation phase (P-value<0.001, FIG. 2A), higher peak thrombin (P-value=0.0015, FIG. 2B), shorter time to peak thrombin (P-value<0.001, FIG. 2C), and faster rate of thrombin generation (P-value=0.003, FIG. 2D). There was no statistically significant difference in total thrombin generation between the groups (FIG. 2E). Next investigated was the association between plasma thrombin generation and AD severity (as assessed by SCORAD) and skin barrier dysfunction (as measured by TEWL) in children. It was found that children with moderate-to-severe AD had increased total thrombin generation (P-value=0.043, FIG. 3A) compared to those with mild disease. In addition, having moderate-to-severe AD was associated with increased peak thrombin (P-value=0.05, Table 2).

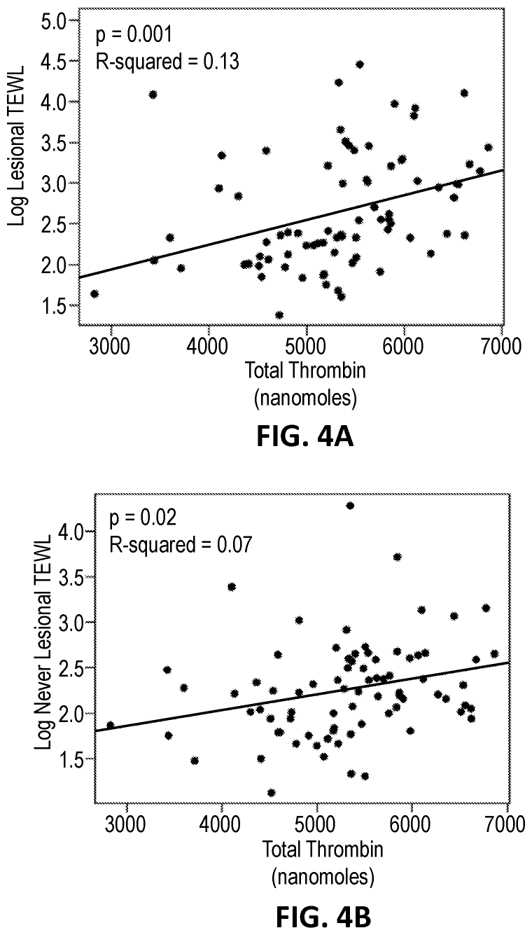

Increased AD skin barrier dysfunction in children was also significantly associated with increased plasma thrombin generation. Specifically, there was a significant association between increased lesional TEWL and increased total plasma thrombin (β=0.30, P-value=0.001, FIG. 4A) and increased peak plasma thrombin (β=0.16, P-value=0.01). Interestingly, increased total plasma thrombin generation was associated with TEWL even in areas of skin that had never been affected by AD (i.e., never-lesional skin) (β=0.17, P-value=0.02, FIG. 4B).

TABLE 2
Associations between atopic dermatitis (AD) outcomes and thrombin generation
assay, fibrinogen, platelet count, and thrombophilic genotypes

| | Moderate-Severe AD | | Lesional TEWL | | Never lesional TEWL | |
|---|---|---|---|---|---|---|
| | Odds | | Beta- | | Beta- | P- |
| Thrombin generation assay | Ratio | P-value | coefficient | P-value | coefficient | value |
| Total thrombin (nanomoles) | 2.10 | 0.02 | 0.30 | 0.00 | 0.17 | 0.02 |
| Lag phase/time to thrombin clot | 0.99 | 0.84 | −0.02 | 0.32 | 0.01 | 0.71 |
| initiation (seconds) | | | | | | |
| Peak thrombin (nanomoles/liter) | 1.49 | 0.05 | 0.16 | 0.01 | 0.03 | 0.53 |
| Time to peak thrombin (seconds) | 0.97 | 0.48 | −0.02 | 0.18 | 0.00 | 0.89 |
| Velocity index/rate of thrombin | 1.01 | 0.14 | 0.00 | 0.13 | 0.00 | 0.91 |
| generation (nanomoles/second) | | | | | | |
| Fibrinogen | 1.00 | 0.30 | −0.10 | 0.04 | −0.04 | 0.28 |
| Platelets | 1.00 | 0.32 | 0.00 | 0.68 | 0.00 | 0.46 |

Lesional, but not Never-Lesional, TEWL is Associated with Plasma Fibrinogen

Thrombin cleaves fibrinogen to initiate fibrin clot formation. Fibrin is crucial for stable crosslinking of platelet thrombi. Fibrin also directly promotes key inflammatory events, including leukocyte activation and migration. Thus, it was determined whether the above associations seen with thrombin were related to plasma fibrin(ogen). Of note, the fibrin(ogen) nomenclature indicates the potential for role for fibrinogen and/or fibrin, particularly in processes where the respective roles have not been elucidated. Total plasma fibrinogen was inversely associated with lesional TEWL (β=−0.099, P-value=0.039). Plasma fibrinogen was not associated with any of the other AD biomarkers (Table 2), including never-lesional biomarkers. Since fibrinogen was only associated with lesional TEWL, this may reflect fibrinogen's role as an acute phase reactant. Moreover, this marginal negative association may represent plasma fibrin(ogen) deposition at lesional sites, thereby increasing local inflammation. Like fibrinogen, platelets are also activated by thrombin. Platelet activation by thrombin leads to clot formation and release of proinflammatory cytokines and chemokines stored within platelet granules, which can impact leukocyte trafficking and activity. Therefore, it was also assessed whether the described AD associations with thrombin were due to increases in circulating platelet counts in children. There was no association between platelet count and the above markers of AD in children (Table 2).

### Pediatric AD is not Associated to Two Common Genetic, Thrombophilic Disorders

Because of the findings that thrombin generation correlates with disease severity and barrier dysfunction in children with AD, it was identified if this might be due to the presence of the two most common prothrombotic genetic mutations: Factor V Leiden (FFL, rs6025) or Prothrombin G20210A (rs1799963). Carriers of risk alleles for these polymorphisms have increased thrombin generation potential and an increased risk for thromboembolic events. Factor V Leiden (FFL) mutation (rs6025) is the most common genetic thrombophilic disorder. No association was found between the risk allele frequency of either factor V Leiden or prothrombin G20210A and AD in children (Table 2). Moreover, the allele frequencies in the subset were no different than the general population (Table 3). These findings suggest that above associations seen between thrombin generation and AD markers are not linked to the two most common thrombophilic genetic disorders.

TABLE 3
Allele frequencies in the study cohort and the general
population for two common thrombophilic
disorders in children: Factor V Leiden (rs6025) and
Prothrombin G20210a (rs1799963)

| | Study | | Study | dbSNP |
|---|---|---|---|---|
| | Cohort | dbSNP | Cohort | (African |
| | (Whites) | (European) | (Blacks) | American) |
| rs6025 | 0.02 | 0.03 | 0.01 | 0.01 |
| (T allele freq) | | | | |
| rs1799963 | 0.02 | 0.01 | 0.01 | 0.00 |
| (A allele freq) | | | | |

### Thrombin Inhibition Attenuates Disease Development in a Mouse Model of AD

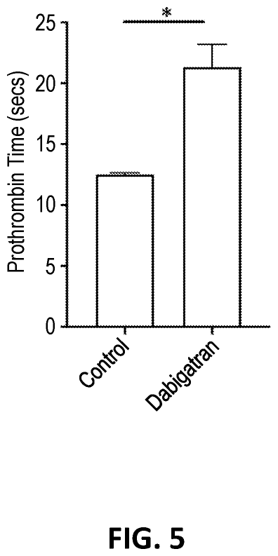

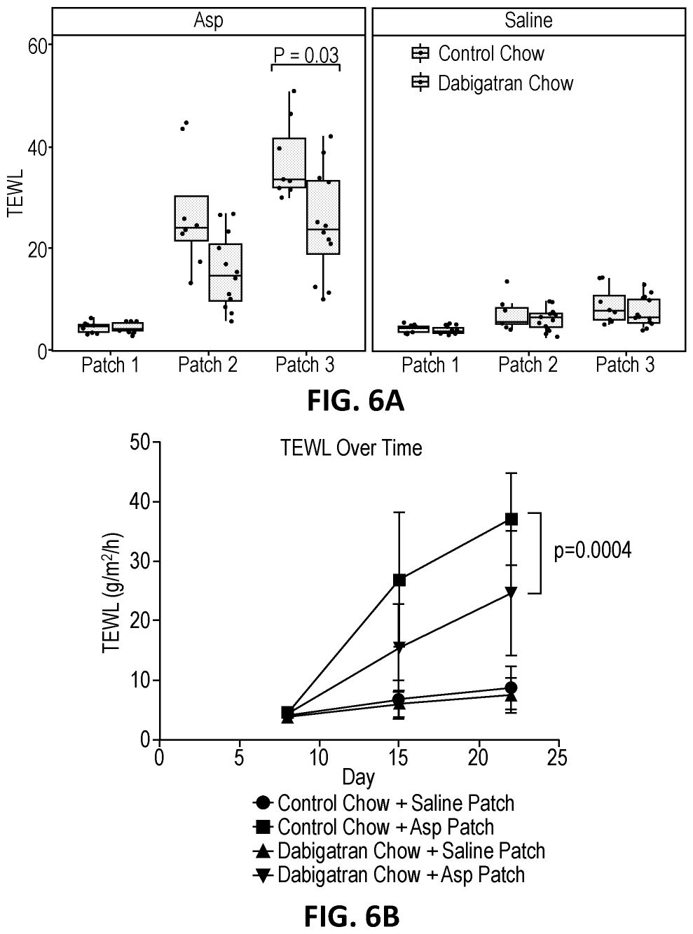

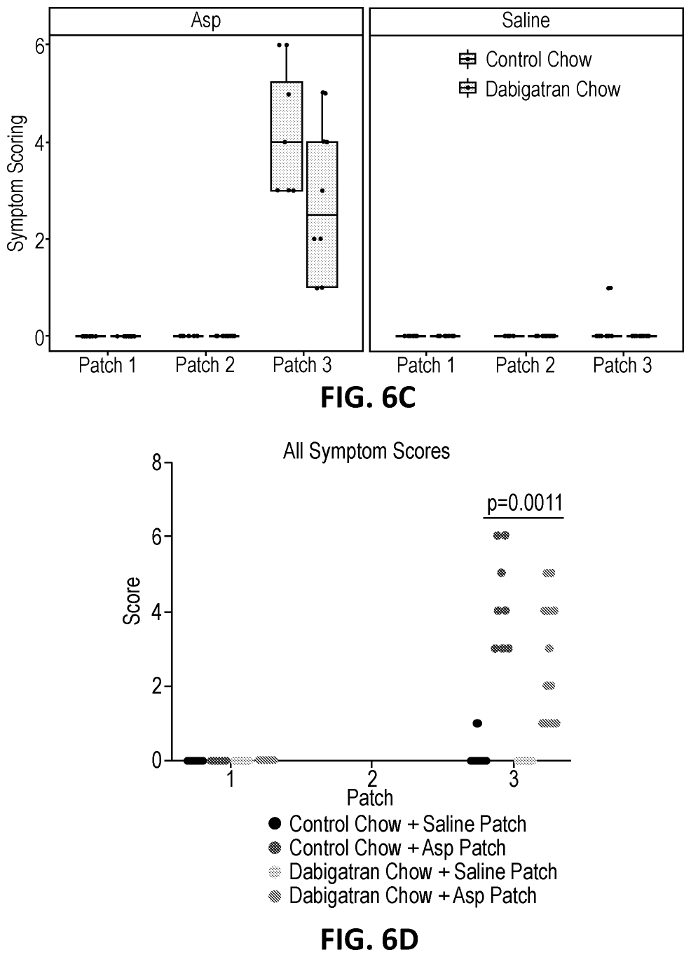

To determine if the association between increased thrombin generation of and AD severity is merely a marker of disease, or if thrombin is mechanistically coupled to AD pathogenesis, an established mouse model of AD was used. Mice were given either chow with dabigatran, a direct thrombin inhibitor (Antonijevic et al., Curr Drug Metab, 2017), incorporated at a dose of 7.5 mg/kg of chow or control diet ad lib. Dabigatran administration increased prothrombin time compared controls (FIG. 5), resulting in a similar change seen in human subjects receiving therapeutic dabigatran doses. Mice (n=40) then underwent an established model of 3 repeated cutaneous allergen patchings to induce an AD-like phenotype (Brandt et al., J Immunol, 2013; Zhang et al., J Immunol, 2014; Stevens et al., Nat Commun, 2020). After 3 patches, mice treated with dabigatran had decreased TEWL (P-value=0.03) compared with control mice (FIGS. 6A and 6B) and a trend towards decreased symptom severity (P-value=0.07) (FIGS. 6C and 6D).

### Complete PAR-1 Deficiency is not a Determinant of AD in Mice

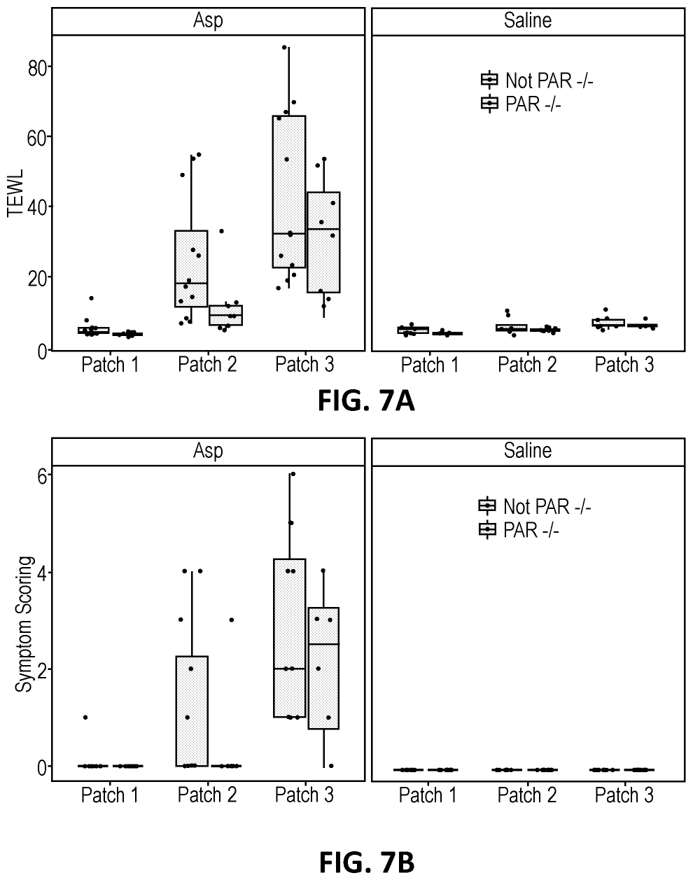

Thrombin can initiate cell signaling via activation of protease activated receptor-1 (PAR-1), which is expressed by keratinocytes and multiple immune cells known to play a role in AD. To determine if thrombin is mechanistically coupled to AD by activation of PAR-1, PAR-1^{-/-} mice and controls with AD were challenged. There was no difference in PAR-1^{-/-} mice compared with controls (n=32, FIGS. 7A-7B). Thus, while PAR-1 may be involved in both barrier disruptive and protective pathways, a complete PAR-1 deficiency does not impact

### Fibrin(Ogen) Promotes AD Pathogenesis

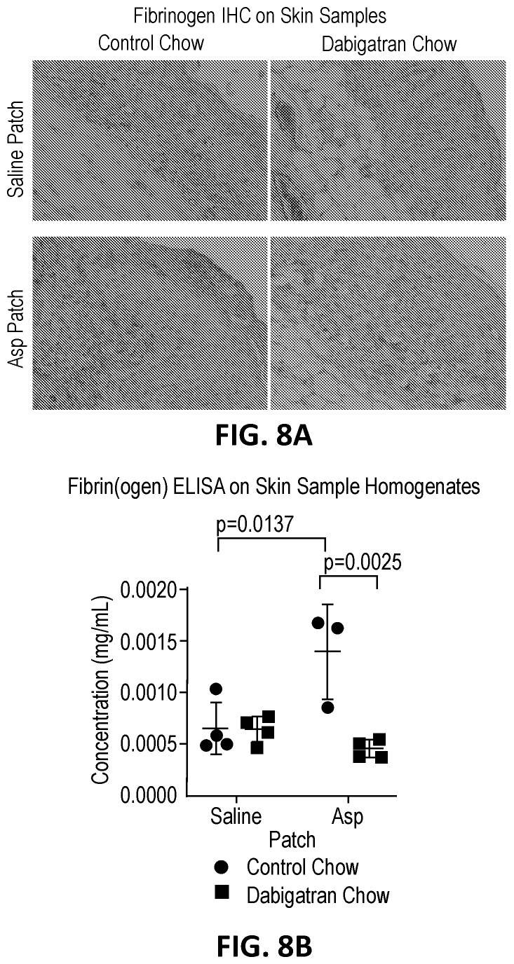

It was then tested whether fibrinogen, a major thrombin substrate, impacted AD development in the mouse model (Brandt et al.; Zhang et al.; Stevens et al.). First, immunohistochemistry was performed on skin samples from mice in each experimental group in the AD model. Dabigatran administration resulted in markedly decreased fibrin(ogen) staining in the Asp group compared with controls (FIG. 8A). Next, a fibrin(ogen) enzyme linked immunosorbent assay was performed on whole skin homogenates using a random sampling of mice from each group. Mice in the Asp patch group that received dabigatran had significantly lower fibrin(ogen) (P-value=0.0025, FIG. 8B). In fact, the amount of fibrin(ogen) in the skin of these animals was comparable to that in unchallenged mice.

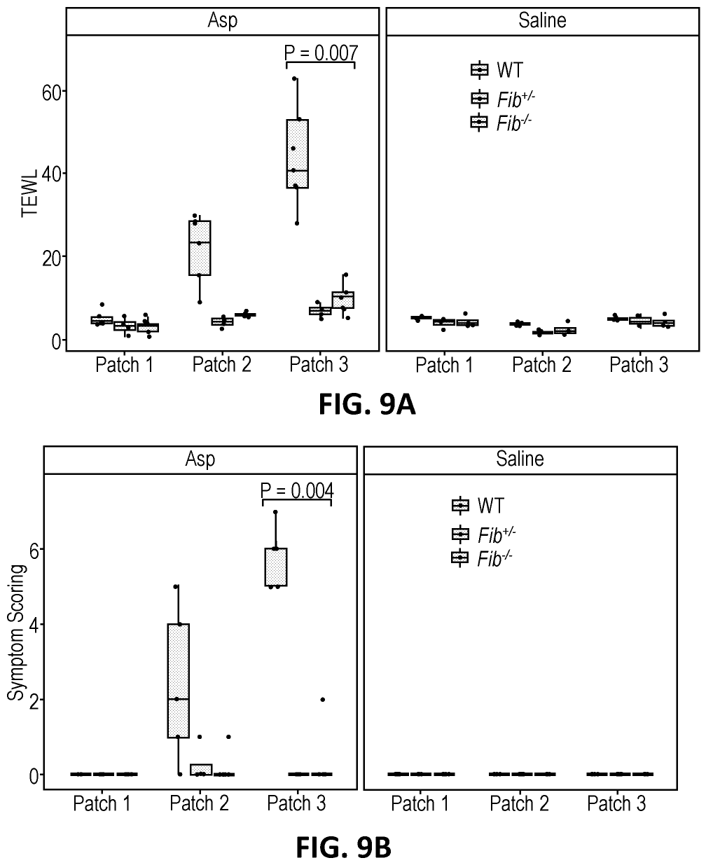

To directly determine fibrinogen's direct impact on AD pathogenesis, mice with complete (Fib^{-/-}) and partial (Fib^{+/-}) fibrinogen deficiency and wildtype controls (FibWT) were enrolled in the mouse model of AD (n=30). After 3 patches Fib^{-/-} and Fib^{+/-} mice had significantly decreased disease development (FIGS. 9A-9B) compared with controls. Fib^{-/-} and Fib^{+/-} had decreased TEWL compared with control mice (P-value=0.0079 and P-value=0.016, respectively; FIG. 9A). Moreover, Fib^{-/-} and Fib^{+/-} had decreased median disease severity compared with control mice (P-value=0.0092 and P-value=0.014, respectively; FIG. 9B). There was no difference between Fib^{-/-} and Fib^{+/-} with respect to TEWL or symptom score. Notably the TEWL and disease severity scores observed in both Fib^{-/-} and Fib^{+/-} were similar to unchallenged controls. It is also notable that Fib^{+/-} mice carry fibrinogen levels that are half normal, a level that does not impair hemostasis whatsoever, but nevertheless resulted in a significant attenuation in AD severity.

### SUMMARY

Taken together, this example demonstrates the mechanistic contributions of thrombin and fibrinogen to AD pathogenesis. These results suggest that thrombin and fibrinogen can be used as effective biomarkers for determining AD presence and/or severity and also can be used as therapy targets for patients with AD.

### Example 2. Topical Formulation of a Thrombin Inhibitor for Treating Atopic Dermatitis

Direct thrombin inhibitor bivalirudin was formulated in a lyophilized formulation containing 50 mg/mL of bivalirudin, 25 mg/mL of mannitol, and NaOH for pH adjustment to 5-6. In aqueous solutions, bivalirudin solubility was influenced by pH but was not affected by temperature and the addition of mannitol. The lyophilized bivalirudin was reconstituted with 5 mL water to a concentration of 50 mg/mL. This formulation prevented bivalirudin precipitate because (1) the aqueous solution contained a buffer and has a pH greater than the isoelectric point of bivalirudin; (2) bivalirudin salt was added to the aqueous solution to form a bulk solution which was transferred to one or more vessels; and (3) the bulk solution was dried. The bulk solution had a final pH of about 4 to about 7 and therefore buffer was added to the bivalirudin topical formulation to make a final pH of about 7.

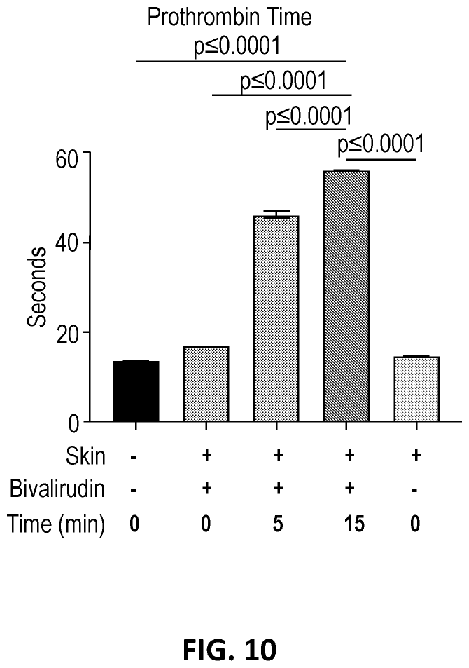

Reconstituted bivalirudin was applied to ex vivo mouse skin samples for 0, 5, or 15 minutes then repeatedly washed, the samples were homogenized, and finally a prothrombin time was run on the homogenized skin samples. This experiment showed that a topically applied thrombin inhibitor can be absorbed by the skin and retain its function, suggesting a use in treating skin disorders. FIG. 10 illustrates absorption of topically applied bivalirudin by measuring prothrombin time. Results show that the topical application of a direct thrombin inhibitor (e.g., reconstituted bivalirudin) to skin is absorbed and retains function.

A topical formulation of the reconstituted bivalirudin was also developed for ease of application. For topical formulation, an oil-in-water emulsion comprising surfactants, stearic acid, thickener, and preservatives was used. This final formulation, with added carbomers to thicken, maintained a pH of around 7. While this formulation used bivalirudin, other thrombin inhibitors may be used.

Taken together, these data strongly support that thrombin and fibrinogen inhibition can be used as a new treatment strategy for AD using topical formulations.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for treating atopic dermatitis in a subject, the method comprising:
administering an effective amount of a composition to a subject in need thereof, wherein the composition comprises a therapeutic agent inhibiting plasma thrombin and a pharmaceutically acceptable carrier.

2. The method of claim 1, wherein the subject is a human pediatric patient having atopic dermatitis.

3. The method of claim 1 or claim 2, wherein the subject has moderate-to-severe atopic dermatitis.

4. The method of claim 3, wherein the subject is diagnosed as having moderate-to-severe atopic dermatitis using a SCORing Atopic Dermatitis (SCORAD) index scoring system and/or an Eczema Area and Severity Index (EASI) scoring system.

5. The method of any one of claims 1-4, wherein the subject has an increased level of total plasma thrombin and/or peak plasma thrombin as compared to a reference value for total plasma thrombin and/or peak plasma thrombin.

6. The method of claim 5, wherein the level of total plasma thrombin or peak plasma thrombin is measured by a thrombin generation assay (TGA).

7. The method of any one of claims 1-6, wherein the subject has an increased level of plasma fibrinogen as compared to a reference value for plasma fibrinogen.

8. The method of claim 7, wherein the level of plasma fibrinogen is measured by an immunohistochemistry (IHC) staining assay or an enzyme-linked immunosorbent assay (ELISA).

9. The method of any one of claims 1-8, wherein the subject has an increased level of transepidermal water loss (TEWL) as compared to a reference value for TEWL.

10. The method of any one of claims 1-9, wherein the level of transepidermal water loss (TEWL) is measured using a Dermalab instrument.

11. The method of any one of claims 1-10, wherein the subject has a skin barrier dysfunction.

12. The method of any one of claims 1-11, wherein the subject is free of a genetic thrombophilic disorder associated with a Factor V Leiden (FFL, rs6025) mutation or a Prothrombin G20210A (rs1799963) mutation.

13. The method of any one of claims 1-12, wherein the therapeutic agent is a direct thrombin inhibitor.

14. The method of claim 13, wherein the direct thrombin inhibitor is selected from the group consisting of dabigatran, bivalirudin, argatroban, hirudin, lepirudin, desirudin, argatroban, inogatran, melagatran, and ximelagatran.

15. The method of any one of claims 1-14, wherein the composition is administered orally, parenterally, or and topically.

16. The method of claim 15, wherein the composition comprises about 40-60 mg/ml bivalirudin, about 20-30 mg/ml mannitol, and a pH of about 7, and wherein the composition is administered to the subject topically.

17. The method of claim 16, wherein the composition is prepared by reconstituting a lyophilized formulation with water or a buffering agent, and wherein the lyophilized formulation comprises about 40-60 mg/ml bivalirudin, about 20-30 mg/ml mannitol, and a pH of about 5-6.

18. A method for diagnosing atopic dermatitis in a subject, the method comprising:
(a) measuring a level of a biomarker in one or more biological samples from a subject suspected of having atopic dermatitis;
(b) comparing the level of the biomarker in the biological sample(s) with a reference value for the biomarker; and
(c) determining presence or severity of atopic dermatitis in the subject based on the comparing results of (b), wherein an elevated level of the biomarker in the biological sample(s) relative to the reference value is indicative that the subject has atopic dermatitis or moderate-to-severe atopic dermatitis;
wherein the biomarker is thrombin, fibrinogen, transepidermal water loss (TEWL), or a combination thereof.

19. The method of claim 18, further comprising treating the subject who is determined as having atopic dermatitis in step (c) a therapeutic agent inhibiting plasma thrombin.

20. The method of claim 18 or claim 19, wherein the biomarker comprises total plasma thrombin, peak plasma thrombin, or a combination thereof.

21. The method of any one of claims 18-20, wherein the biomarker comprises plasma fibrinogen.

22. The method of any one of claims 18-21, wherein the one or more biological samples comprise a blood sample, a skin sample, or a combination thereof.

23. The method of claim 22, wherein the biomarker comprises total plasma thrombin and/or peak plasm thrombin, and wherein the level of the total plasma thrombin and/or peak plasma thrombin is measured in a blood sample of the subject.

24. The method of claim 23, wherein the level of the total plasma thrombin and/or peak plasma thrombin is measured by a thrombin generation assay (TGA).

25. The method of claim 22, wherein the biomarker comprises fibrinogen, and wherein the level of fibrinogen is measured in a skin sample of the subject.

26. The method of claim 25, wherein the level of the fibrinogen is measured by an immunohistochemistry (IHC) staining assay or an enzyme-linked immunosorbent assay (ELISA).

27. The method of claim 22, wherein the biomarker comprises transepidermal water loss (TEWL).

28. The method of claim 27, wherein the level of transepidermal water loss (TEWL) is measured using a Dermalab instrument in a skin sample of the subject.

29. The method of any one of claims 18-28, further comprising assessing atopic dermatitis severity in a subject who has been determined to have atopic dermatitis in step (c) using a SCORing Atopic Dermatitis (SCORAD) index scoring system and/or an Eczema Area and Severity Index (EASI) scoring system.

30. The method of any one of claims 18-29, wherein the subject is a human pediatric subject.

31. The method of any one of claims 18-30, wherein the subject has a skin barrier dysfunction.

32. The method of any one of claims 18-31, wherein the subject is free of a genetic thrombophilic disorder associated with a Factor V Leiden (FFL, rs6025) mutation or a Prothrombin G20210A (rs1799963) mutation.

33. The method of any one of claims 19-32, wherein the therapeutic agent is a direct thrombin inhibitor.

34. The method of claim 33, wherein the direct thrombin inhibitor is dabigatran, bivalirudin, argatroban, hirudin, lepirudin, desirudin, argatroban, inogatran, melagatran, or ximelagatran.

35. The method of any one of claims 19-34, wherein the therapeutic agent is administered orally, parenterally, or topically.

36. A lyophilized formulation, which is lyophilized from an aqueous solution comprising about 40-60 mg/ml bivalirudin, about 20-30 mg/ml mannitol, and having a pH of about 5-6.

37. The lyophilized formulation of claim 36, wherein the aqueous solution comprises 50 mg/ml of bivalirudin and 25 mg/ml mannitol.

38. The lyophilized formulation of claim 36 or claim 37, wherein the aqueous solution comprises NaOH for pH adjustment.

39. A method for treating a skin disorder in a subject, the method comprising:
(i) providing the lyophilized formulation of any one of claims 36-38;
(ii) reconstituting the lyophilized formulation with water or a buffering agent to produce a final aqueous formulation comprising about 40-60 mg/ml bivalirudin, about 20-30 mg/ml mannitol, and having a pH of about 7; and
(iii) administering the final aqueous formulation topically to the subject.

40. The method of claim 39, wherein the final aqueous formulation comprises 50 mg/ml bivalirudin and 25 mg/ml mannitol, and has a pH of about 7.

41. The method of claim 39 or claim 40, wherein the subject is a human patient having atopic dermatitis; optionally wherein the human patient is a pediatric patient.
